(19) United States
(12) Patent Application Publication
(10) Pub. No.: US 2005/0191373 A1
(43) Pub. Date: Sep. 1, 2005

(54) Agent for the prevention and/or treatment of septicemia

(75) Inventors: Naohito Ohno, Tokyo (JP); Susumu Masuda, Noda-Shi (JP)

Correspondence Address: BIRCH STEWART KOLASCH & BIRCH, PO BOX 747, FALLS CHURCH, VA, 22040-0747, US

(73) Assignee: Kikkoman Corporation, Noda-Shi (JP)

(21) Appl. No.: 11/058,633

(22) Filed: Feb. 16, 2005

(30) Foreign Application Priority Data
Feb. 17, 2004 (JP) 2004-039859

(51) Int. Cl. A61K 47/00; A61K 35/78

(52) U.S. Cl. 424/757; 424/439

(57) ABSTRACT

The purpose of the invention is to provide an agent for the prevention or treatment of septicemia caused by the infection of microorganism. The present invention relates to: (1) an agent for the prevention and/or treatment of septicemia caused by the infection of microorganism, comprising soybean extract or soybean fermented product as an effective; (2) An agent for the prevention and/or treatment of septicemia caused by the infection of microorganism, comprising isoflavone as an effective component; and (3) Food and drink, medicine, or cosmetics, comprising the agent for the prevention and/or treatment according to the above (1) or (2). The agent according to the present invention, which has a high suppressing activity of the abnormal production of TNF-α, is useful in the fields of medicines, cosmetics and foods, especially for the profundus mycosis patient.

### TECHNICAL FIELD

The present invention relates to an agent for the prevention and/or treatment of septicemia caused by the infection of microorganism.

### BACKGROUND ART

Septicemia is Systemic Inflammatory Response Syndrome (SIRS), which is caused by the infection of microorganisms such as bacteria or fungi. The term “septicemia” in this specification includes also septic shock and multi-organ [symbol] deficiency that will very likely occur subsequently. The incidence of the septicemia is a serious problem in medical sites. The septicemia is considered to develop by the production of various kinds of inflammatory cytokines such as a tumor necrosis factor α (TNF-α) in response to the invasion of endotoxin derived from bacteria into blood. The TNF-α is a typical cytokine that is produced by activated macrophages, which are sensitized by bacteria, viruses, parasites, etc. It is known that the abnormal production of TNF-α caused by the infection of bacteria is closely related to the medical conditions of septicemia. The TNF-α is involved in various immuno and inflammatory reactions and is deeply related to the medical conditions of various inflammatory disorders.

Substance, which can suppress or inhibit the abnormal production of TNF-α, is useful as an agent for the prevention, improvement or treatment of the various inflammatory disorders including septicemia. Agents for suppressing the production of TNF-α, which are extracted from plant materials such as Japanese basil and tea, are known as described in the documents (1)-(6) listed below.
(1) The Japanese Patent Application Publication No. Hei 7(1995)-215884;
(2) The Japanese Patent Application Publication No. Hei 7 (1995)-258097;
(3) The Japanese Patent Application Publication No. Hei 10 (1998)-72361;
(4) The Japanese Patent Application Publication No. 2001-48804;
(5) The Japanese Patent Application Publication No. 2001-238638; and
(6) The Japanese Patent Application Publication No. 2003-26586,

There is, however, still a strong desire for the development of a new substance that can be orally administered to suppress the production of TNF-α, and has safety guaranteed through its long history of being served as a food.

Profundus mycosis, which recently occurs with a higher incidence, is an infectious disease wherein organs or tissues in a body are invaded by fungi. It will be a fatal disease for a patient in the immunosuppressive condition due to cancers, transplantation of organs, acquired immunodeficiency syndrome and the like. Furthermore, total body care accompanying anti-tumor and anti-bacteria chemotherapies, or surgical and emergency medical treatments have increased the number of patients who are susceptible to the profundus mycosis. There is a problem that the profundus mycosis patents will very likely suffer septic shock since their susceptibility to bacterial endotoxin has been increased due to β-glucan produced by the fungi.

It is therefore very important from a medical point of view to develop an agent for the prevention and/or treatment of septicemia.

### SUMMARY OF THE INVENTION

The present inventors have studied hard to solve the above problems, and finally found that isoflavone derived from soybean extract, especially soybean fermented product has an activity of suppressing the abnormal production of TNF-α.

Thus, the present invention is based on the above findings and relates to an agent for the prevention or treatment of septicemia as described below.
(1) An agent for the prevention and/or treatment of septicemia caused by the infection of microorganism, comprising soybean extract or soybean fermented product as an effective component.
(2) An agent for the prevention and/or treatment of septicemia caused by the infection of microorganism, comprising isoflavone as an effective component.
(3) Food and drink, medicine, or cosmetics, comprising the agent for the prevention and/or treatment according to claim 1 or 2.

### ADVANTAGES OF THE INVENTION

The agent for the prevention and/or treatment of septicemia according to the present invention has a high activity of suppressing the abnormal production of TNF-α, and is therefore useful in the fields of food, medicine and cosmetics. The agent according to the present invention is especially useful for the profundus mycosis patients who are liable to develop septicemia.

### BRIEF DESCRIPTION OF DRAWING

FIG. 1 shows the results of the measurement of TNF-α concentration in serum, as described in Experiment in the present specification. An error bar in the figure means a standard error.

### BEST MODE FOR CARRYING OUT THE INVENTION

As described above, the microorganisms include bacteria, fungi and the like that are involved in septicemia.

The soybean extract used as the effective component in the present invention may be prepared by extracting soybean materials such as intact soybean, defatted soybean, soybean germ, soybean meal, and separated soybean protein with an appropriate solvent. The solvent used in the extraction has no limitation, including usual polar solvent, non-polar solvent, for example, an organic solvent or a mixed solvent containing thereof such as that of water and the organic solvent. The organic solvent includes lower alcohol such as ethanol, methanol, propanol, butanol; ethers such as diethyl ether; halogenated hydrocarbons such as chloroform; nitrites such as acetonitrile; esters such as ethyl acetate; ketones such as acetone; hexane; dimethylsulfoxide; dimethylformamide, etc. Ethanol, methanol and ethyl acetate are preferable in view of operability. Two or more kinds of solvent may be used as a mixture thereof. Although there is no limitation on a mixing ratio between water and the organic solvent, the mixture is desired to comprise the organic solvent at ratio of 20% or more, especially between 40˜90%.

Water-soluble foreign substances may be removed from the soybean materials by extracting them with water or hot water prior to the extraction with the solvent In such case, the residue obtained by the water extraction is collected and subjected to the extraction with the solvent. The extraction process is preferably repeated several times in order to improve extraction efficiency. After the completion of the extraction, the solvent containing the effective component is collected by means of, for example, aspiration filtration to give the soybean extract according to the present invention in a liquid state. A process such as the extract with the solvent and a treatment with a synthetic adsorbent or ion-exchange resin may be further carried out in order to concentrate and purify the effective component in the soybean extract.

The “soybean fermented product” used as the effective component in the present invention may be prepared by treating the soybean materials with microorganisms or enzymes. The microorganism includes Aspergillus, Lactobacillus, yeast, etc. The soybean fermented product may be preferably subjected to a conventional extraction or concentration process in order to concentrate and purify the effective component in the soybean fermented product.

In the process of preparation of the extract of the soybean fermented product with lactobacillus, the occurrence of immature smell may be prevented in advance by crushing the soybean material with hot water at 80° C. or more so as to inactivate lipoxygenase contained therein. Soybean milk having no immature smell is therefore obtained by removing soybean residue from the resulting soy bean slurry, “go”, and then subjected to UHT sterilization. Lactobacillus is then inoculated in the soybean milk sterilized with heating to start lactic acid fermentation. Any Lactobacillus may be used in the present invention, such as Lactobacillus helveticus, Lactobacillus bulgaricus, Lactobacillus casei, Lactobacillus acidophillus, Streptococcus thermophillus, and Lactococcus lactis. The above Lactobacillus, which has been cultured in the soybean milk or other suitable culture medium, is added to the above soybean milk in an amount of about 0.5˜3.0% by weight of the soybean milk.

The fermentation conditions may be optionally determined depending on the kind of Lactobacillus, the concentration of the soybean milk and the like. Lactic acid fermentation is usually carried out for 10˜24 hours at 20˜42° C. to produce fermented-soybean milk. The resulting fermented soybean milk is dried by, for example, spray-dry to give the soybean fermented product. Soy cake or residue obtained as a by-product in the production of soy sauce may be used as the soybean fermented product a swell. The resulting soybean fermented product comprises isoflavones, saponin, saccharides, peptides and the like.

The soybean fermented product is preferably crushed finely prior to the extraction. The resulting extract of the soybean fermented product may be used in any form such as that including the solvent, its concentrate, or a dry product obtained by removing the solvent, the dry product being preferable in view of storage stability and safety.

Isoflavone is the most preferable effective component in the agent according to the present invention. It may be obtained from plant materials such as soybean by, for example, extracting or concentrating the fermented product prepared by treating the soybean materials with the microorganisms or enzymes. It may alternatively be chemically synthesized. It may be in a form of aglycon or glycoside. The aglycon of isoflavone includes genistein, daidzein and glycitein, and its glycoside includes genistin, daidzin and glycitin.

As shown by the Example in the present specification, the soybean fermented product comprising isoflavone in a high concentration significantly inhibits the increase of the concentration of TNF-α in blood under infectious conditions by the bacteria or fungi. Thus, the soybean extract, the soybean fermented product and isoflavone are advantageously used as the agent for the prevention and/or treatment of septicemia caused by the infection of microorganism, and may be comprised in food and drink, medicine, or cosmetics for mammalians including human.

The agent according to the present invention may be usually administered orally. Thus, an effective amount of the agent may be comprised in an edible form, for example, food and drink; dietary supplement foods for a particular purpose such as food for patients, milk for pregnant and lactating women, modified milk powder for infants, food for elderly people, and food for specified health use; enteral nutritional supplement, quasi-drug or medicine. The above agent may alternatively be taken by means of an enteral nutrition intake method, in which elemental nutrition diet, low-residual diet, or natural liquid diet is fed orally, or via gastric fistula, jejunum fistula or a tube inserted through a nose into a stomach (Internal medicine outline, Vol. 6 “obesity and clinical nutrition” p. 289-307, 1995 (in Japanese) Nakayama Bookstore).

The agent of the present invention may be formulated in any form such as pill, capsule, granulate, powder and liquid, depending on the purpose of administration, the kind and conditions of disorders, and the kind of a subject to be administered. It may be directly administered or as a mixture with feeding stuff or beverage. A dose of the effective component of the present invention may vary depending on the purpose of administration, the kind and conditions of disorders, and the kind of the subject to be administered, being usually 1˜2,000 mg/kg, preferably 5˜1,000 mg/kg, more preferably 10˜200 mg/kg. isoflavone has been taken in dairy life.

The present invention will be more specifically described with reference to the following example, which should not be construed as limiting the scope of the invention.

### EXAMPLE

The following experiment uses a model mouse with bacterial β-glucan load for the administration of endotoxin in order to confirm the advantage of the agent according to the present invention on septic shock. The model mouse is injected with the bacterial β-glucan so that its susceptibility to endotoxin will be elevated just like in the condition of profundus mycosis. Upon injection of lipopolysaccharide (LPS) as endotoxin into the model mouse, the abnormal production of TNF-α is initiated, and septic shock will be subsequently induced. The specific procedures and results of the experiment are described below.

Experiment

1) Test-Animal:

Male DBA/2 mice (6 weeks old on purchase; Charles River Laboratories Inc., Japan) were used. The mice were divided into groups and preliminarily bred for one week prior to the experiment. During preliminary breeding and the experiment, they were bred under a usual lighting cycle and fed freely with a commercial solid feeding stuff (Oriental Yeast Co., Ltd., MF Feeding Stuff and water.

2) The Agent According to the Present Invention:

Extract of the soybean fermented product (“SoyAct Y”, Kikkoman Corp.)

3) Experiment Procedures:

A solution (0.2 ml) containing β-glucan (Sigma Co.) in physiological saline was intraperitoneally administered at a dose of 200 μg/mouse to prepare a profundus mycosis-infected model mouse.

The test mice were divided into two groups of (1) isoflavone group, and (2) control group, each of which consisted of 10 mice. To the mice of the isoflavone group and the control group were orally administered by means of a gastric catheter a suspension (0.2 ml) of isoflavone (60 mg/shot) in carboxymethylcellulose (CMC) solution, and CMC solution (0.2 ml), respectively. Physiological saline solution (50 μg/mouse) containing LPS (Sigma Co.) was then intravenously injected to the mice of the two groups. After 90 min., blood was collected and let to stand for one hour at a room temperature. The collected blood was then centrifuged and its supernatant was diluted appropriately. The resulting supernatant sample was subjected to ELISA measuring kit (Amersham Pharmacia Co., [(m) TNF-α], mouse, ELISA system RPN2718) to detect the concentration of TNF-α in the supernatant.

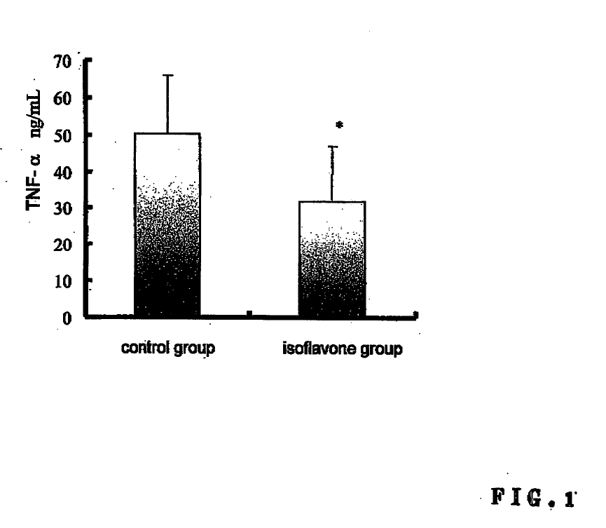

The results are shown in FIG. 1. It is observed that the isoflavone group showed a significant suppression of the increase of the concentration of TNF-α in the blood compared to the control group (Risk Rate: <1%, Fisher test). This result demonstrated that the oral administration of isoflavone suppressed the abnormal production, of TNF-α caused by UPS, and would prevent the systemic septic shock.

### Formulation Example

Drink (11 kg) was prepared by mixing 5 g of SoyAct Y, 50 g of sugars, 15 g of honey, 1 g of ascorbic acid, 0.5 g of citric acid, an appropriate amount of a flavoring ingredient, and the rest of water. It was then sterilized and aseptically divided equally into a 100 ml bottle. The resulting drink comprising isoflavone as the effective component is used in the prevention and/or treatment of septicemia caused by the infection of microorganism.

1. An agent for the prevention and/or treatment of septicemia caused by the infection of microorganism, comprising soybean extract or soybean fermented product as an effective component.

2. An agent for the prevention and/or treatment of septicemia caused by the infection of microorganism, comprising isoflavone as an effective component.

3. Food and drink, medicine, or cosmetics, comprising the agent for the prevention and/or treatment according to claim 1.

4. Food and drink, medicine, or cosmetics, comprising the agent for the prevention and/or treatment according to claim 2.
